(19) United States
(12) Patent Application Publication
(10) Pub. No.: US 2014/0207194 A1
(43) Pub. Date: Jul. 24, 2014

(54) System Comprising a Bone Plate and a Bone Screw

(71) Applicant: Dietmar Wolter, Ahrensburg (DE)

(72) Inventor: Dietmar Wolter, Ahrensburg (DE)

(21) Appl. No.: 14/237,932

(22) PCT Filed: Aug. 9, 2012
(86) PCT No.: PCT/EP2012/065607

§ 371 Date: Feb. 11, 2014

(30) Foreign Application Priority Data
Aug. 11, 2011 (EP) 11177230.7

(51) Int. Cl. A61B 17/80

(52) U.S. Cl. CPC A61B 17/8033
USPC 606/288

(57) ABSTRACT

A system comprises a bone plate and a bone screw. The bone plate is provided with a through-hole, which extends from an upper face to a bone-side lower face of the bone plate. A lip is formed in the through-hole, which lip protrudes from the curved surface of the through-hole and extends in the circumferential direction of the through-hole. The bone screw has a head thread, which is designed to reshape the lip in the through-hole in order to form a threaded connection. The lip has a step arranged between the curved surface of the through-hole and a central region of the lip, which step extends along the lip. The system has the advantage that a bone screw can easily cut into the lip.

### BACKGROUND

The invention relates to a system comprising a bone plate and a bone screw. The bone plate is provided withy a through-hole that extends from an upper face of the bone plate to a lower face thereof situated toward the bone. A lip is formed in the through-hole, which lip protrudes from the curved surface of the through-hole and extends in the circumferential direction of the through-hole. The bone screw has a head thread, which is designed to reshape the lip in the through-hole in order to form a threaded connection.

Bone plates of this kind are designed to be connected to a bone. They can be used, for example, to stabilize a bone after a fracture. For this purpose, the bone plate is positioned such that it extends across the fracture site, and it is then secured on the bone fragments. The fracture site is thereby immobilized, and the bone is able to heal. The bone plate can also be used for other purposes and, for example, can be an element of an endoprosthesis to be connected to a bone.

To secure the bone plate on the bone, a bone screw is inserted into the through-hole, said bone screw being provided with a thread both on the shank and also on the head. The shank of the screw penetrates so far into the bone substance that the head of the screw enters the through-hole of the bone plate. The head of the bone screw is dimensioned such that its external diameter is greater than the smallest diameter of the lip. As the bone screw is screwed in further, the lip is deformed, such that a threaded connection forms between the head of the bone screw and the lip in the through-hole of the bone screw. Since there is a threaded connection both between the shank of the bone screw and the bone substance and also between the head of the screw and the bone plate, a secure connection is produced between the bone plate and the bone. Since the threaded connection between the head of the bone screw and the bone plate is formed only by a deformation process when the bone screw is screwed in, it is not necessary for the bone screw to be screwed at a specific predefined angle into the bone plate. Instead, the angle can be freely chosen within certain limits. This gives the surgeon a high level of flexibility during the operation.

In previous bone plates, the head thread of the bone screw, shortly after the initial engagement, has to displace quite a large amount of material in order to form the thread. The engagement of the head thread in the lip is thus made difficult.

### SUMMARY

A system comprises a bone plate and a bone screw, in which system the bone screw can easily engage in the lip.

The lip has a step arranged between the curved surface of the through-hole and a central area of the lip, which step extends along the lip.

A number of terms will first be explained. A lip designates an area where the material thickness is reduced compared to the material thickness of the bone plate. The lip protrudes from the curved surface of the through-hole in the direction of the center of the through-hole. The lip has a longitudinal extent along the circumference of the through-hole. The lip extends preferably without interruption about the entire circumference of the through-hole. The step can extend along the entire length of the lip. The lip has a lip top surface directed toward the upper face of the bone plate, and a lip underside directed toward the lower face of the bone plate, said top surface and underside each having an extent in the circumferential direction and an extent in the radial direction. The lower face of the bone plate lies on the bone when the bone plate is fitted.

When the lip is provided with a step, this means that, starting from the center and moving outward in the radial direction, the lip has a greater gradient in the area of the step than in the area of the central portion. Starting from the center of the through-hole, the step follows the central portion in the peripheral direction. Gradient designates the angle that the lip top surface or lip lower face encloses with the plane extending transversely with respect to the axial direction of the through-hole.

By means of the step, it is made possible that the lip has, in the central area, a portion in which the material thickness is small. The bone screw, which engages in this portion of the lip, initially has to displace only a small amount of material. Nevertheless, directly after the first engagement, there is still a secure guide, such that the bone screw, when screwed in further, moves along a defined path. It is only when the bone screw advances into the area of the step that more material has to be displaced. The threaded connection between the head of the bone screw and the lip is thus formed in two stages. In a first stage, the head thread engages in an area of small material thickness, such that the initial engagement takes place easily and with slight force being applied. Only when the head thread has advanced into the area of the step is so much material displaced that the threaded connection acquires sufficient stability.

The lip can be provided with a step on the lip top surface. Alternatively or in addition to this, a step can be provided on the lip underside.

For some embodiments, the peripheral end of the step merges into the curved surface of the through-hole. The gradient of the step is then preferably at least 20°, preferably at least 30°, more preferably at least 45° smaller than the gradient of the curved surface of the through-hole. The curved surface of the through-hole can have a gradient of 90°. To ensure that sufficient space remains above the lip for the head of the bone screw, such that the bone screw can be screwed in at different angles without colliding with the curved surface of the through-hole, the gradient of the curved surface can also be less than 90°.

Alternatively, the lip can comprise a peripheral area, which extends between the step and the curved surface of the through-hole. The gradient in the peripheral area is less than the gradient of the step. If the step is at a distance from the curved surface of the though-hole, the bone screw can be inserted at different angles into the through-hole without coming into collision with the curved surface.

When the head thread has been advanced into the area of the step, a secure threaded engagement already exists. There is therefore no longer any danger of the head thread slipping on the lip, even if the head thread now has to engage in the steeper step. The surgeon feels the step as an appreciably increased resistance while he is screwing in the screw. When the gradient of the step is sufficiently great, from this point a limited rotation angle is sufficient to form the final threaded connection. This increases the user friendliness, since an indication that the bone screw is to be rotated further, for example by 90° or 180°, is easier for the surgeon to follow than it is to observe a certain predefined torque. In a preferred embodiment, the gradient of the step is therefore greater than 45°, preferably greater than 60°, more preferably greater than 75°.

The central area of the lip serves primarily to permit an easy engagement of the head thread, while the head thread finds its actual hold in the parts of the lip that lie farther to the outside. For the first engagement, it is sufficient if the central area makes up only a small part of the lip. Preferably, the distance from the central end of the lip to the central end of the step is not greater than 50%, preferably not greater than 30%, more preferably not greater than 20% of the distance from the central end of the lip to the curved surface of the through-hole.

The gradient of the step should be much greater than the gradient in the central area. Preferably, the gradient of the step is at least 20°, preferably at least 30°, more preferably at least 45° greater than the gradient of the central area. The gradient in the central area can, for example, be between 0° and 40°, preferably between 20° and 30°. The step can, for example, have a gradient of between 60° and 90°. If the lip comprises a peripheral area, the gradient there can likewise lie between 0° and 40°, preferably between 20° and 30°.

To be sufficiently easily deformable, the lip, even at its thickest point, is preferably much thinner than the bone plate. Preferably, the thickest point of the lip extends over not more than 80%, preferably not more than 60%, more preferably not more than 50% of the length of the through-hole.

It is advantageous if the bone screw screwed into the bone plate protrudes as little as possible above the bone plate, since otherwise there is the danger of surrounding tissue being irritated. If the lip is arranged in the (lower) half of the through-hole adjacent to the lower face of the bone plate, there is space above the lip in order for the head of the bone screw to be recessed in the bone plate. The (upper) half of the through-hole adjacent to the upper face of the bone plate is then formed by the curved surface of the through-hole, in other words is free of the lip.

The bone plate, with the lip in the through-hole, can be formed in one piece. Alternatively, it is possible that an area surrounding the through-hole is inserted in the form of an inlay into the bone plate. The inlay with the lip can firstly be formed as a separate part and can then be connected to the bone plate. To make the threaded connection easier to form, the inlay can be made from a softer material than the bone plate.

To allow the surgeon the option of using conventional bone screws in which the head is not provided with a thread, the curved surface of the through-hole can be widened outward above the lip. This part of the curved surface can then form an abutment surface for a conventional screw head. In cross section, the widening can have the shape of a segment of a circle, for example.

The invention also relates to a system composed of such a bone plate and of a bone screw. The bone screw has a head thread which is designed to reshape the lip in the through-hole, in order to form a threaded connection. To permit secure fastening to the bone, the bone plate generally has a plurality of through-holes. Accordingly, the system can also comprise a plurality of bone screws.

The material deformation taking place during the formation of the threaded connection should cause the least possible amount of chip abrasion. Tests have shown that the chip abrasion is kept low if the screw head has a conical curved surface and the angle that the curved surface encloses with the axis of the screw is between 19° and 28°.

### BRIEF DESCRIPTION OF THE DRAWINGS

The invention is described below by way of example on the basis of advantageous embodiments and with reference to the attached drawings, in which:

FIG. 1 shows a view of a bone plate from above;

FIG. 2 shows a cross section through FIG. 1 along the line I-I;

FIG. 3 shows an enlarged view of detail A from FIG. 1;

FIGS. 4 to 7 show different embodiments of lips;

FIGS. 8 and 9 show the view from FIG. 2 in other embodiments; and

FIG. 10 shows a bone screw intended for the bone plate.

### DETAILED DESCRIPTION

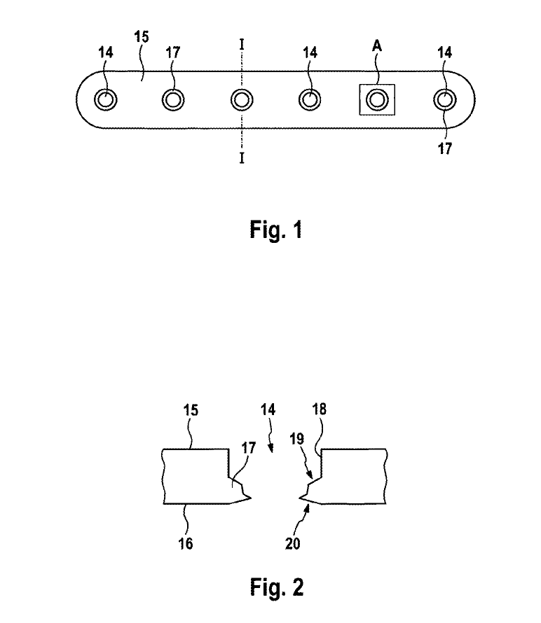

In accordance with FIGS. 1 and 2, a bone plate according to the invention has six through-holes 14 that extend from an upper face 15 of the bone plate to a lower face 16 of the bone plate. A lip 17 is formed in each through-hole 14, which lip 17 protrudes from the curved surface 18 in the direction of the center of the through-hole 14. The lip 17 is a continuous lip, which extends without interruption about the entire circumference of the through-hole. The lip 17 has a lip top surface 19 directed toward the upper face 15 of the bone plate, and a lip underside 20 directed toward the lower face 16 of the bone plate. The lip 17 ends with the lower face 16 of the bone plate, such that as much space as possible remains above the lip 17 in order to receive the screw head.

The bone plate is intended to be placed with the lower face 16 onto a bone, such that the bone plate extends across a fracture site of the bone. After the bone has been returned to the correct position, the bone plate is connected to the bone fragments. The bone is then fixed in this position and is able to heal.

To connect the bone plate to the bone, bone screws 31 are used, as are shown in FIG. 10. The bone screws 31 have a shank 27, which is provided with a bone thread 28, and a screw head 29 whose outer surface has a head thread 30 of smaller pitch. The curved surface of the screw head 29 encloses an angle of 25° with the axis of the screw. The bone screw is screwed into the bone until the screw head 29 enters the through-hole 14. The angle that the bone screw encloses with the axis of the through-hole can be freely chosen between about 0° and 15°. When the bone screw is now screwed in further, the head thread 30 of the bone screw 31 comes into engagement with the lip 17. The lip 17 is deformed, and a threaded connection is formed between the head 29 of the bone screw 31 and the lip 17. To make formation of the threaded connection easier, the bone screw 31 can be made from a harder material than the bone plate. For example, the bone plate can be produced from pure titanium, while the bone screw 31 is made from the titanium alloy TiAl6V4.

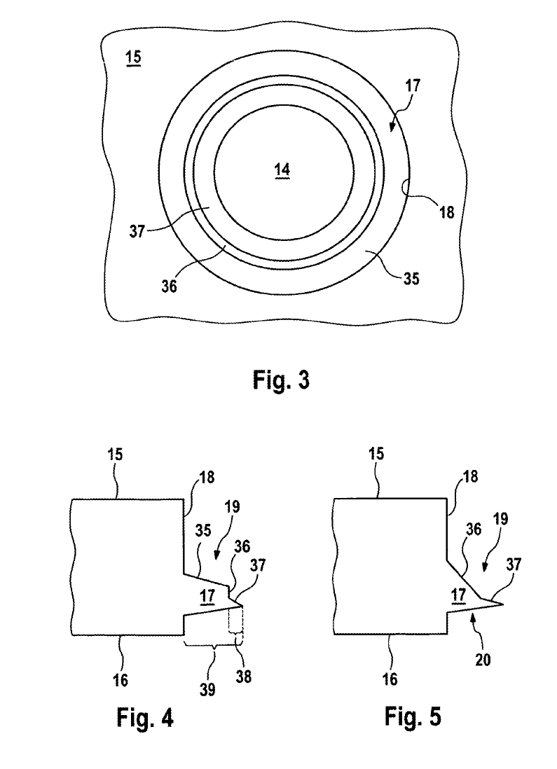

According to FIG. 3, the lip 17 comprises a peripheral area 35, a step 36, and a central area 37. The lip and the areas 35, 36, 37 extend about the entire circumference of the through-hole 14. In the peripheral area 35 and in the central area 37, the gradient of the lip top surface is small and measures about 15°, for example. In the area of the step 36, which connects the central area 37 and the peripheral area 35, the gradient is substantially greater and measures 75°, for example.

When a bone screw 31 is screwed into the through-hole 14, the head thread 30 first comes into engagement with the central area 37 of the lip 17. The lip is thin in the central area 37, such that only a small amount of material is displaced upon penetration of the head thread 30. The surgeon, who is screwing in the screw, notices hardly any increased resistance. Upon further screwing, the head thread 30 penetrates farther into the central area 37 and approaches the step 36. When the head thread 30 reaches the step 36, the thread is already formed to the extent that it provides a secure guide for the bone screw. When the head thread 30 bears on the step 36, there is an increase in the resistance that has to be overcome upon further screwing-in of the screw, since a larger amount of material now has to be reshaped. Starting from the point of increased resistance, the bone screw 31 is screwed in a further 90°, such that a threaded connection forms that extends about the entire circumference of the bone screw 31. The end state is thereby reached, and the head thread 30 is locked securely in the lip 17.

FIG. 4 shows an embodiment in which the step 36 has a gradient of 90°. The step 36 is therefore parallel to the curved surface 18 of the through-hole 14. The central area 37 of the lip 17 makes up only the smaller part of the lip 17. The distance 38 between the central end of the lip and the step 36 measures only about a quarter of the distance 39 between the central end of the lip 17 and the curved surface 18 of the through-hole 14.

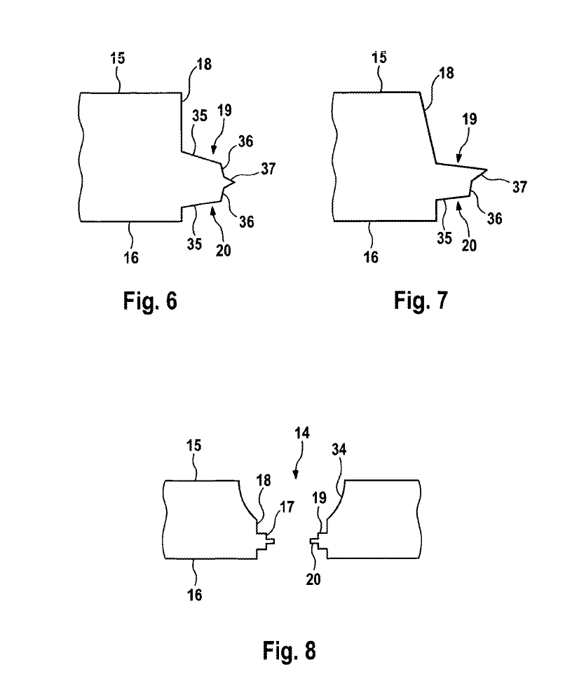

In the embodiment in FIG. 5, the lip 17 is composed only of a central area 37 and of a step 36. The step 36 extends from the peripheral end of the central area 37 to the curved surface 18 of the through-hole 14. In FIG. 6, both the lip top surface 19 and also the lip underside 20 are provided with a step 36. The step 36 is adjoined, both on the lip top surface 19 and also on the lip underside 20, by a peripheral area 35 in which the gradient is much less than at the step 36. In the embodiment in FIG. 7, only the lip underside 20 has a step 36. The curved surface 18 of the through-hole 14 is inclined slightly outward, such that more space remains for receiving the screw head.

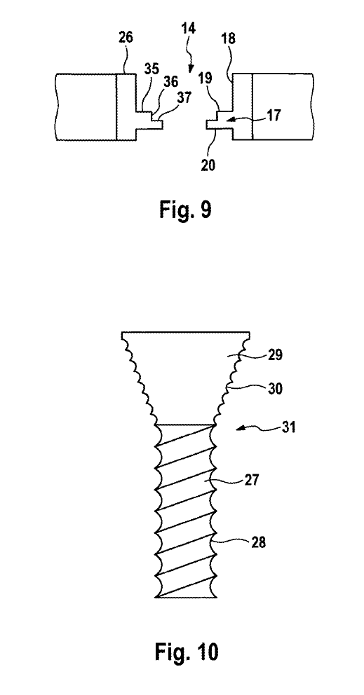

FIG. 9 shows an embodiment in which an inlay 26 is inserted into the bone plate. The inlay 26 is made from a softer material than the bone plate and forms the lip 17 and the surround of the through-hole 14. The lip comprises a central area 37 and a peripheral area 35, in which areas the gradient is 0°. The step 36 has a gradient of 90°.

In FIG. 8, the curved surface 18 above the lip 17 has a widening 34 which, in cross section, has the shape of a segment of a circle. With this embodiment, there is the option of using conventional bone screws in which the head does not have a thread. The widening 34 then forms an abutment face for a bone screw with a hemispherical head, such that the bone screw can be inserted at different angles into the through-hole 14.

1. A system comprising a bone plate and a bone screw, wherein the bone plate is provided with a through-hole that extends from an upper face of the bone plate to a lower face thereof situated toward the bone, wherein a lip is formed in the through-hole, which lip protrudes from the curved surface of the through-hole and extends in the circumferential direction of the through-hole, and wherein the bone screw has a head thread, which is designed to reshape the lip in the through-hole in order to form a threaded connection, characterized in that the lip has a step arranged between the curved surface of the through-hole and a central area of the lip, which step extends along the lip.

2. The system as claimed in claim 1, characterized in that the lip has a top surface which is provided with a step.

3. The system as claimed in claim 1, characterized in that the lip has an underside which is provided with a step.

4. The system as claimed in claim 1, characterized in that the lip comprises a peripheral area, which extends between the step and the curved surface of the through-hole.

5. The system as claimed in claim 1, characterized in that the step has a gradient and the gradient of the step is greater than 45°.

6. The system as claimed in claim 1, characterized in that a distance is defined between a central end of the lip and a central end of the step and the distance between the central end of the lip and the central end of the step is not greater than 50% of the distance between the central end of the lip and the curved surface of the through-hole.

7. The system as claimed in claim 1, characterized in that the step has a gradient and the central area has a gradient and the gradient of the step is at least 20° greater than the gradient of the central area.

8. The system as claimed in claim 1, characterized in that the through hole has a length and the lip extends in an axial direction by not more than 80%, of the length of the through-hole.

9. The system as claimed in claim 1, characterized in that the lip is arranged in a half of the through-hole adjacent to the lower face of the bone plate.

10. The system as claimed in claim 1, characterized in that an area surrounding the through-hole is of an inlay inserted into the bone plate.

11. The system as claimed in claim 1, characterized in that the bone screw has a head and an as with a conical curved surface, and in that the curved surface encloses an angle of between 19° and 28° with the axis of the screw.

12. The system as claimed in claim 1, characterized in that the step has a gradient and the gradient of the step is greater than 60°.

13. The system as claimed in claim 1, characterized in that the step has a gradient and the gradient of the step is greater than 75°.

14. The system as claimed in claim 1, characterized in that a distance is defined between a central end of the lip and a central end of the step and the distance between the central end of the lip and the central end of the step is not greater than 30% of the distance between the central end of the lip and the curved surface of the through-hole.

15. The system as claimed in claim 1, characterized in that a distance is defined between a central end of the lip and a central end of the step and the distance between the central end of the lip and the central end of the step is not greater than 20% of the distance between the central end of the lip and the curved surface of the through-hole.

16. The system as claimed in claim 1, characterized in that the step has a gradient and the central area has a gradient and the gradient of the step is at least 30% greater than the gradient of the central area.

17. The system as claimed in claim 1, characterized in that the step has a gradient and the central area has a gradient and the gradient of the step is at least 45° greater than the gradient of the central area.

18. The system as claimed in claim 1, characterized in that the through hole has a length and the lip extends in an axial direction by not more than 60% of the length of the through-hole.

19. The system as claimed in claim 1, characterized in that the through hole has a length and the lip extends in an axial direction by not more than 50% of the length of the through-hole.

20. The system as claimed in claim 2, characterized in that the lip has an underside which is provided with a step.
